(19) United States
(12) Patent Application Publication
(10) Pub. No.: US 2025/0221842 A1
(43) Pub. Date: Jul. 10, 2025

(54) Inflatable constriction ring device mountable to vacuum erection device providing airtight system for constricting venous outflow from penis

(71) Applicant: Maqsood Jafri, Glen Carbon, IL (US)

(72) Inventor: Maqsood Jafri, Glen Carbon, IL (US)

(73) Assignee: Magqsood Jafri, Glen Carbon, IL (US)

(21) Appl. No.: 18/445,741

(22) Filed: Jan. 10, 2024

(51) Int. Cl. A61F 5/41

(52) U.S. Cl. CPC A61F 5/41; A61F 2005/412; A61F 2005/414; A61F 2005/415

(57) ABSTRACT

The apparatus described is an inflatable constriction ring device to constrict the venous outflow from penis. The inflatable constriction ring is used in combination with vacuum erection device (VED), which induces erection and the ring constricts the venous outflow at the base of the penis to maintain erection.

The distal end of constriction ring device is mountable on the proximal end of vacuum erection device to make a sealable contact with the vacuum erection device. The constriction ring is configured to make sealable contact with the user's skin around the penis thus creating an airtight system. The constriction ring is attached to the tubing of the inflation/deflation pump, which is used to inflate the constriction ring.

### FIELD

This technology as disclosed herein relates generally to medical devices and, more particularly, to medical devices to address erectile dysfunction.

### BACKGROUND

Erectile dysfunction is a medical condition that impacts men from the early part of middle age and approximately 50% of males have the problem by the age of 70. The medical disorder can be manifested as the inability to obtain and/or sustain an erection. Various devices have been utilized to sustain an erection including various constriction devices. However, many of the devices available are cumbersome and or don't adequately address the problem, particularly in the more severe cases of the disorder. A more practical and effective medical device is needed to address this common dysfunction.

### SUMMARY

The technology as disclosed herein includes an apparatus for constricting the venous outflow from penis to address erectile dysfunction. The technology includes an annular inflatable constriction ring that annularly surrounds a penis, the constriction ring contacts sealably with the user's skin, and can also be mounted sealably on vacuum erection device (VED). The mounted inflatable ring thus forms the air-tight system when used in combination with existing vacuum erection device. The activation of vacuum erection device induces erection, the constriction ring is inflated that constricts the base of penis to restrict venous outflow thus maintaining erection. The constriction ring is detached from the vacuum erection device after the erection is achieved. The inflation/deflation pump is attachable to the outer aspect of the inflatable constriction ring to inflate the constriction ring.

The features, functions, and advantages that have been discussed can be achieved independently in various implementations or may be combined in yet other implementations further details of which can be seen with reference to the following description and drawings.

These and other advantageous features of the present technology as disclosed will be in part apparent and in part pointed out herein below.

### BRIEF DESCRIPTION OF THE DRAWINGS

FIG. 1 is a side view of a vacuum erection device along with a mounted inflatable constriction ring.

FIG. 2 is the bottom view of a coaxial inflatable constriction ring device showing the structure of the mountable ring. The upper part of the outer surface of the ring is mounted on the vacuum erection device.

FIG. 3 Bottom view of inflatable ring showing another implementation of technology where the upper part of outer solid ring is mountable to vacuum erection device and inflatable ring for encircling the penis is disposed inside the outer solid ring.

FIG. 4A, 4B, 4C Bottom view of inflatable ring showing attachment of vibratory units and loops for engaging vibratory units

### DETAILED DESCRIPTION OF DIAGRAMS

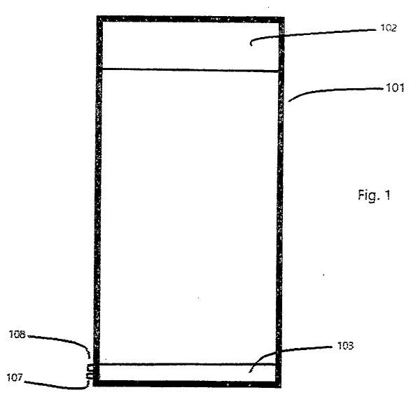

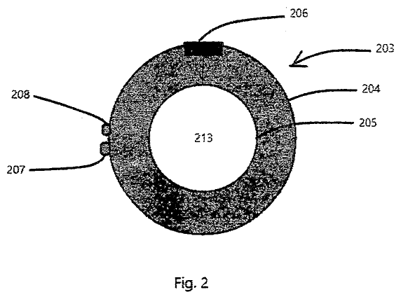

FIG. 1 illustrates the front view of the vacuum erection device 101 with the mounted inflatable ring 103, vacuum pump 102 of the vacuum erection device 101.

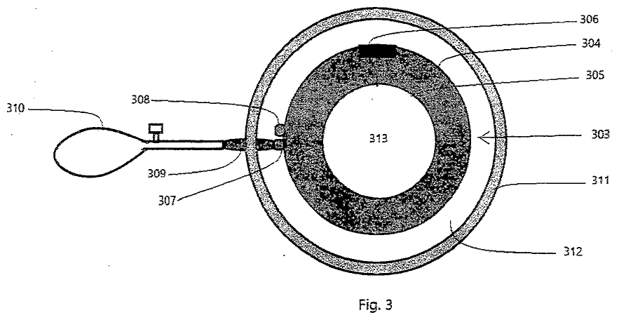

FIG. 2 illustrates the bottom view of the inflatable constriction ring 203, outer inelastic but flexible surface 204 is mounted on the vacuum erection device. Inner inflatable surface 205 constricts the penis after inflation, locking mechanism 206 that is engaged after the ring is wrapped around the penis, inflation/deflation port 207 for receiving the tubing of inflation/deflation pump, and release valve 208 to deflate the ring after use. Penis occupies position 213

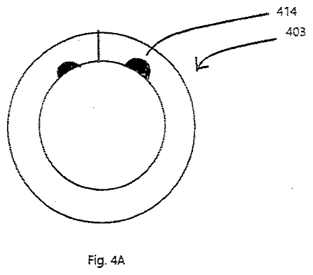

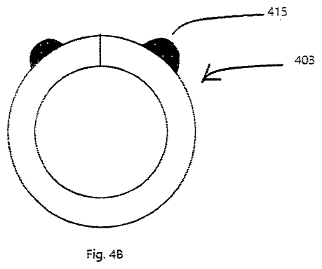

FIG. 3 illustrates the bottom view of inflatable constriction ring 303 that is housed in the interior of solid outer ring 311. Inflatable ring 303, outer inelastic surface 304, inner inflatable surface 305, locking mechanism 306, inflation/deflation port 307, release valve 308, airtight port 309 that engages with the inflation deflation port 307, Inflation/deflation pump 310 connectable to airtight apparatus, outer solid outer ring 311 upper part of which is mounted on vacuum erection device. The space between inner surface of outer solid ring 311 and outer surface 304 of inflatable ring 303 is 312. The ring does not form a sealing relationship with the outer hard ring. The penis occupies position 313. The arrangement described for implementation shown in FIG. 3 makes the inflatable constriction ring less bulky and gives more options for sizing the inflatable ring.

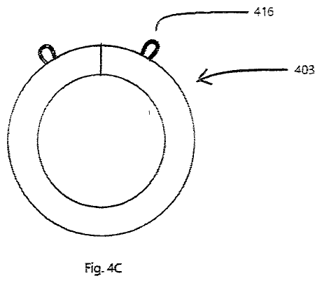

FIGS. 4A, 4B, 4C indicates the bottom view of constriction rings 403. FIG. 4A illustrates the vibratory units 414 attached to the interior of the inner wall of the inflatable ring 403. FIG. 4B shows the vibratory units 415 attached to the outer exterior of outer wall of inflatable constriction ring 403. FIG. 4C indicates loops 416 for engaging vibratory units attached to the outer wall of the inflatable constriction ring.

### DESCRIPTION

According to the implementation(s) of the present technology as disclosed, various views are illustrated in FIG. 1-4 and like reference numerals are being used consistently throughout to refer to like and corresponding parts of the technology for all of the various views and figures of the drawing.

The device consists of commercially available vacuum erection device (VED) 101 to induce erection and proposed mountable inflatable constriction ring devices 103, 203, 303, and 403 to cause constriction of the outflow of blood from erect penis to maintain erection and impart vibration to the penis in case of inflatable constriction ring 403.

The vacuum erection device 101 with mounted inflatable ring 103 is pressed against pubic skin to make airtight seal thereby allowing the suction in the vacuum chamber to induce erection and making it practicable to inflate the inflatable constriction ring at the base of the erect penis restricting outflow of blood thus maintaining erection. Once the erection is achieved the inflatable constriction ring device is inflated, vacuum chamber is detached from the inflatable ring and removed, the tubing of the inflation pump is detached from the inflatable constriction ring device leaving the inflatable constriction ring device at the base of the erect penis to maintain erection.

In one implementation of technology the outer surface 204 of an inflatable constriction 203 ring is made of flexible inelastic material and inner surface 205 that comes in contact with the penis is made of inflatable material that is compatible with skin. The upper outer inelastic part of 204 is mounted on vacuum erection device 101. The inflation/deflation pump (not shown in FIG. 2) that is connectable to port 207 and is communicable with the interior bladder of the ring 203 is configured to push a flow of air or liquid to fill the bladder and expand at least the inner surface of the inflatable ring to thereby reduce its diameter and apply an inward pressure against the penis to constrict blood flow. The pump is removed after inflating the ring. The inflation/deflation pump can be incorporated in the vacuum pump 102 of vacuum erection device. An intermediate tapering bushing can be interposed between the VED 101 and ring 103 with narrow end engaging the inflatable constriction ring in order to facilitate application of smaller size ring.

Another implementation of technology that consists of outer solid ring 311 and inner inflatable ring. The upper part of the outer solid ring is mounted on a vacuum erection device and the inflatable ring 303 is housed inside the outer solid ring. The inflatable constriction ring 303 is attachable to the inflation deflation pump 310 through the air-tight port 309 incorporated in the solid ring 311 that in turn engages port 307 of ring 303. There is space 312 between the outer surface of the ring 304 and outer solid ring 311.

Yet in another implementation of technology the vacuum erection device is placed in a grooved inflatable ring. The groove is located on the surface facing the VED opposite to the surface of the ring that makes a sealing contact with the pubic skin. The vacuum chamber of VED makes an airtight seal with the grooved part of the ring.

In another implementation of technology the inflatable ring is made of two layered components, outer layer and inner layer. The partially stretchable outer component forms the cover for the inner inflatable balloon component. The surface of the outer cover facing penis is stretchable and remaining surfaces of outer component are made non-stretchable by reinforcing with stiffer elements thus directing the constrictive force inwards towards penis.

In another implementation of technology small vibrators are attached to the interior of the outer component that makes contact with the penis and impart vibration to penile nerves. Such vibrators can also be positioned on the exterior of the outer component or loops are provided on the exterior of the outer component for securing vibrators after the ring is inflated and vacuum erection device is removed.

The inflatable constriction ring has inflation/deflation pump 310 that is attached by tubing to airtight port 309. The airtight port 309 is in turn connectable to inflation/deflation port 307 of ring 303. The inflatable ring is inflated by using pump 310 to cause constriction of the ring 303. The constriction ring also has a separate pressure release valve 308 to deflate the ring after use.

After inflating the inflatable constriction rings optimally, the tubing of the pump (not shown in FIG. 1, 2, 4) is detached from the inflatable ring port 207 for ring shown in FIG. 2 and airtight port 309 in case of ring shown in FIG. 3. The air-tight port 309 is also disengaged from the inflation/deflation port 307 of ring 303 and outer solid ring 311 is thus removed.

One implementation of technology is that inflation/deflation pump 310 for inflation of inflatable constriction ring device is incorporated in the distal end of the vacuum erection device alongside the vacuum pump 102, which may make the use easier for some patients. The tubing connecting the pump with the inflatable ring is placed external to the vacuum chamber (not shown in diagram)

One implementation of technology is a locking mechanism incorporated into inflatable constriction ring design. The ring is split at a point along its circumference to allow the ring to be opened up and be wrapped around the penis and locked in position thus avoiding the need to slide the ring over the penis that may be difficult in case of a semi-engorged penis.

The locking mechanism may be incorporated in the ring wall with the underlying layer of the inflatable part of the ring in order to exert uniform constriction pressure on penis.

The various implementations and examples shown above illustrate a method and apparatus for forming an airtight system and constricting blood flow. A user of the present method and apparatus may choose any of the above implementations, or an equivalent thereof, depending upon the desired application. In this regard, it is recognized that various forms of the subject constriction method and apparatus could be utilized without departing from the scope of the present technology and various implementations as disclosed.

As is evident from the foregoing description, certain aspects of the present implementation are not limited by the particular details of the examples illustrated herein, and it is therefore contemplated that other modifications and applications, or equivalents thereof, will occur to those skilled in the art. It is accordingly intended that the claims shall cover all such modifications and applications that do not depart from the and scope of the present implementation(s). Accordingly, the specification and drawings are to be regarded in an illustrative rather than a restrictive sense.

Certain systems, apparatus, applications or processes are described herein as including a number of modules. A module may be a unit of distinct functionality that may be presented in software, hardware, or combinations thereof. When the functionality of a module is performed in any part through software, the module includes a computer-readable medium. The modules may be regarded as being communicatively coupled. The inventive subject matter may be represented in a variety of different implementations of which there are many possible permutations.

The methods described herein do not have to be executed in the order described, or in any particular order. Moreover, various activities described with respect to the methods identified herein can be executed in serial or parallel fashion. In the foregoing Detailed Description, it can be seen that various features are grouped together in a single embodiment for the purpose of streamlining the disclosure.

The various implementations and examples shown above illustrate a method and apparatus for addressing erectile dysfunction. A user of the present technology as disclosed may choose any of the above implementations, or an equivalent thereof, depending upon the desired application. In this regard, it is recognized that various forms of the subject erectile dysfunction device could be utilized without departing from the scope of the present invention.

Other aspects, objects and advantages of the present technology as disclosed can be obtained from a study of the drawings.

1. An apparatus for addressing erectile dysfunction consisting of:
an annular pliable constriction ring used in combination with a vacuum erection device causing constriction at the base of a penis to prevent venous outflow;
a seal at a proximal end of the ring is configured to sealably contact a user's skin.

2. The apparatus recited as in claim 1 wherein a seal at a distal end of the ring is configured to sealably mount on a proximal end of vacuum erection device chamber.

3. The apparatus recited as in claim 1 wherein the annular pliable constriction ring structure is inflatable.

4. The apparatus recited in claim 3 further consists of:
an inflation deflation pump integrated in the distal end of vacuum erection device;
a tubing runs along the outer side of the wall of vacuum erection device chamber wherein the tubing communicably connects the pliable constriction ring structure with the inflation pump.

5. The apparatus as recited in claim 1 wherein a groove in the distal end of pliable ring structure is configured to receive the proximal end of the vacuum erection device chamber to sealably contact with the ring.

6. The apparatus for addressing erectile dysfunction comprising:
an outer inelastic solid ring and inner coaxial annular pliable constriction ring;
a seal at the proximal end of outer solid ring is configured to sealably contact user skin;
a seal at the distal end of the outer solid ring configured to sealably mount on the proximal end of vacuum erection device chamber;
an airtight port traverses the outer solid ring.

7. The apparatus as recited in claim 6 wherein the inner pliable constriction ring structure is inflatable.

8. The apparatus recited as in claim 6 wherein the inner pliable coaxial ring has an inflation deflation port connectable to the airtight port traversing the outer solid ring.

8. The apparatus as recited in claim 6 the airtight port communicably connects the inner pliable constriction ring device with inflation deflation pump by tubing running along the outer side of the wall of vacuum erection device chamber.

9. The apparatus as recited in claim 8 the inflation deflation pump is configured to be incorporated in the distal end of the vacuum erection device.

10. The apparatus as recited in claim 3 the pliable constriction ring device structure is configured to attach at least one vibrator to;
an outer wall;
an inner wall.

11. The apparatus as recited in the claim 3 the pliable constriction ring device structure is configured to have a loop or loops to engage vibrator.

12. The apparatus as recited in claim 7 the pliable constriction ring device structure is configured to attach at least one vibrator to;
the outer wall;
the inner wall.

13. The apparatus as recited in the claim 7 the pliable constriction ring device structure is configured to have a loop or loops to engage vibrator.
